(19) United States
(12) Patent Application Publication
(10) Pub. No.: US 2013/0331260 A1
(43) Pub. Date: Dec. 12, 2013

(54) Odour-Control Superabsorbent

(71) Applicant: BASF SE, Ludwigshafen (DE)

(72) Inventors: Olaf Hoeller, Charlotte, NC (US); Volker Braig, Weinheim-Lutzelsachsen (DE); Thomas Daniel, Waldsee (DE); Patrick Hamilton, Charlotte, NC (US)

(21) Appl. No.: 13/908,332

(22) Filed: Jun. 3, 2013

Related U.S. Application Data
(60) Provisional application No. 61/657,068, filed on Jun. 8, 2012.

(51) Int. Cl. B01J 20/22; A61L 15/46; A61L 15/20; B01J 20/26

(52) U.S. Cl. CPC B01J 20/22; B01J 20/267; A61L 15/46; A61L 15/20
USPC 502/402; 564/14; 502/401

(57) ABSTRACT

Superabsorbents comprising fluorofamide exhibit superior odour control without being biocides.

The present invention relates to odour-control superabsorbents comprising fluorofamide, a process for producing such superabsorbents and water-absorbing products that comprise such superabsorbents.

Superabsorbents are known. Superabsorbents are materials that are able to take up and retain many times their weight in water, possibly up to several hundred times their weight, even under moderate pressure. Absorbing capacity is usually lower for salt-containing solutions compared to distilled or otherwise de-ionised water. Typically, a superabsorbent has a centrifugal retention capacity (“CRC”, method of measurement see hereinbelow) of at least 5 g/g, preferably at least 10 g/g and more preferably at least 15 g/g. Such materials are also commonly known by designations such as “high-swellability polymer”, “hydrogel” (often even used for the dry form), “hydrogel-forming polymer”, “water-absorbing polymer”, “absorbent gel-forming material”, “swellable resin”, “water-absorbing resin” or the like. The materials in question are crosslinked hydrophilic polymers, in particular polymers formed from (co)polymerised hydrophilic monomers, graft (co)polymers of one or more hydrophilic monomers on a suitable grafting base, crosslinked ethers of cellulose or starch, crosslinked carboxymethylcellulose, partially crosslinked polyalkylene oxide or natural products that are swellable in aqueous fluids, examples being guar derivatives, of which water-absorbing polymers based on partially neutralised acrylic acid are most widely used. Superabsorbents are usually produced, stored, transported and processed in the form of dry powders of polymer particles, “dry” usually meaning less than 5 wt.-% moisture content (method of measurement see hereinbelow), although forms in which superabsorbents particles are bound to a web, typically a nonwoven, are also known for some applications, as are superabsorbent fibres. A superabsorbent transforms into a gel on taking up a liquid, specifically into a hydrogel when as usual taking up water. By far the most important field of use of superabsorbents is the absorbing of bodily fluids. Superabsorbents are used for example in diapers for infants, incontinence products for adults or feminine hygiene products. Examples of other fields of use are as water-retaining agents in market gardening, as water stores for protection against fire, for liquid absorption in food packaging or, in general, for absorbing moisture.

Processes for producing superabsorbents are also known. The acrylate-based superabsorbents which dominate the market are produced by radical polymerisation of acrylic acid in the presence of a crosslinking agent (the “internal crosslinker”), usually in the presence of water, the acrylic acid being neutralised to some degree in a neutralisation step conducted prior to or after polymerisation, or optionally partly prior to and partly after polymerisation, usually by adding a alkali, most often an aqueous sodium hydroxide solution. This yields a polymer gel which is comminuted (depending on the type of reactor used, comminution may be conducted concurrently with polymerisation) and dried. Usually, the dried powder thus produced (the “base polymer”) is surface crosslinked (also termed surface “post” crosslinked) by adding further organic or polyvalent cationic crosslinkers to generate a surface layer which is crosslinked to a higher degree than the particle bulk. Most often, aluminium sulphate is being used as polyvalent cationic crosslinker. Applying polyvalent metal cations to superabsorbent particles is sometimes not regarded as surface crosslinking, but termed “surface complexing” or as another form of surface treatment, although it has the same effect of increasing the number of bonds between individual polymer strands at the particle surface and thus increases gel particle stiffness as organic surface crosslinkers have. Organic and polyvalent cation surface crosslinkers can be cumulatively applied, jointly or in any sequence.

Surface crosslinking leads to a higher crosslinking density close to the surface of each superabsorbent particle. This addresses the problem of “gel blocking”, which means that, with earlier types of superabsorbents, a liquid insult will cause swelling of the outermost layer of particles of a bulk of superabsorbent particles into a practically continuous gel layer, which effectively blocks transport of further amounts of liquid (such as a second insult) to unused superabsorbent below the gel layer. While this is a desired effect in some applications of superabsorbents (for example sealing underwater cables), it leads to undesirable effects when occurring in personal hygiene products. Increasing the stiffness of individual gel particles by surface crosslinking leads to open channels between the individual gel particles within the gel layer and thus facilitates liquids transport through the gel layer. Although surface crosslinking decreases the CRC or other parameters describing the total absorption capacity of a superabsorbent sample, it may well increase the amount of liquid that can be absorbed by hygiene product containing a given amount of superabsorbent.

Other means of increasing the permeability of a superabsorbent are also known. These include admixing of superabsorbent with fibres such as fluff in a diaper core or admixing other components that increase gel stiffness or otherwise create open channels for liquid transportation in a gel layer.

Frederic L. Buchholz and Andrew T. Graham (Eds.) in: “Modern Superabsorbent Polymer Technology”, J. Wiley & Sons, New York, U.S.A./Wiley-VCH, Weinheim, Germany, 1997, ISBN 0-471-19411-5, give a comprehensive overview over superabsorbents and processes for producing superabsorbents.

When superabsorbents are used in the hygiene sector, they are exposed to bodily fluids such as urine or menses. Such bodily fluids always contain malodourous components such as amines, fatty acids and other organic components which are responsible for unpleasant body odours. A further problem with such hygiene products is that the bodily fluids remain in the hygiene product for a certain time until the hygiene product is disposed of, and bacterial degradation of nitrogenous compounds present in the absorbed bodily fluids, an example being urea in urine, gives rise to ammonia or else other amines which likewise lead to a noticeable odour nuisance. Since correspondingly frequent changing of the hygiene product leads to an appreciable inconvenience and also cost for the user or his or her care persons, hygiene products where this odour nuisance is avoided are of advantage.

Various measures to avoid the odour nuisance are known. Odours can be masked by perfumes; the ammonia which results or amines can be removed by absorption or reaction, and the microbial degradation can be inhibited by means of biocides or urease inhibitors for example. These measures can be applied to the superabsorbent on the one hand and to the hygiene article on the other.

For instance, EP 1 358 894 A1 teaches hygiene articles which include superabsorbent foam and may include a series of odour-preventing additives, in particular anhydride groups, acid groups, cyclodextrins, biocides such as triclosan, surfactants having an HLB value of less than 11, absorbents such as zeolites, clay, activated carbon, silica or activated alumina, micro organisms which act as antagonists to undesirable odour-forming micro organisms, pH buffers or chelating agents. WO 03/002 623 A1, WO 03/028 778 A2 or WO 03/076 514 A2 feature a comprehensive overview of existing measures for avoiding unpleasant odours. WO 2007/104 641 A2 disclose superabsorbents having improved smell-inhibition by addition of keto acids.

U.S. Pat. No. 4,182,881 reports that N-[Diaminophosphinyl]arylcarboxamides are useful as inhibitors of the enzyme urease for treating urinary tract diseases. In particular, this patent relates to compounds of the general formula R—C(O)—NH—P(O)(NH₂)₂ where R is 3-pyridyl, 2-furyl, 2-naphthyl, cinnamenyl, benzyl, phenyl or phenyl substituted by 3- or 4-nitro, 4-halo, 4-amino-, 4-(lower alkoxy), 4-(lower alkyl), 2-methyl, 2,3-dimethyl, 2,4-dimethyl, 2,4,6-trimethyl, 3-trifluoromethyl, 4-cyano, 4-phenyl or 3-phenoxy. One of the examples is N-[Diaminophosphinyl]-4-fluorobenzamide (common name “fluorofamide”, other name N-(Diaminophosphinyl)-4-fluorobenzamide, CAS No. 70788-28-2).

WO 98/26 808 A2 discloses absorbent articles comprising an odour control system that in turn consists of a combination of a material that prevents odour formation and another, odour-absorbing material. Among the material that prevent odour formation, urease inhibitors are named, such as substituted N-(Diaminophosphinyl)benzamides of the formula:

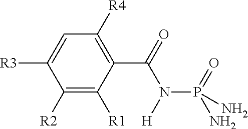

where R1, R2, R3 and R4 are hydrogen, nitro, halogen, amino, C₁-C₄ alkyl, C₁-C₄ alkoxy, trifluormethyl, cyano, phenoxy, phenyl, and mixtures thereof.

For clarification, although fluorofamide is not named in WO 98/26 808 A2: For fluorofamide, R1, R2 and R4 are hydrogen and R3 is fluorine in this formula.

N-(Diaminophosphinyl)benzamides, fluorofamide and its urease inhibiting effect have been the subject of some studies. K. Kobashi, S. Takebe, A. Numata, J. Biochem. 98:1681-1688, 1985 and O. E. Millner, Jr., J. A. Andersen, M. E. Appler, C. E. Benjamin, J. G. Edwards, D. T. Humphrey, and E. M. Shearer, J. Urol., 127:346-350, 1982 report IC50 measurements (the concentration of a compound to inhibit 50% of enzyme activity) of the non-fluorinated analogue of fluorofamide and fluorofamide itself on Proteus mirabiliis and Proteus morganii, respectively. S. H. Phadnis, M. H. Parlow, H. Levy et al., Infect Immun, 64, 905-912, 1996 and H. L. T. Mobley, R. P. Island, Hausinger, Microbiol. Rev. 59, 451, 1995 evaluate the total urease activity versus only surface localised urease activity. A. J. Pope, N. Toseland, B. Rushant, S. Richardson, M. McVey and J. Hills, Digestive Diseases and Sciences, 43, 109-119, 1998 state that fluorofamide has no measurable antibacterial properties for Helicobacter sp. J. M. Bremner and M. J. Krogmeier, Proc. Natl. Acad. Sci. 85, 4601-4604, 1988 report effects of fluorofamide on seed germination in urea-based fertiliser and determined that fluorofamide worked better than its non-fluorinated analogue in most cases.

Despite the advanced state of the art as outlined in the cited prior art, there still is a need for superabsorbents exhibiting improved odour control and for processes for their production. It is an object of this invention to find such a superabsorbent and process.

This object has been solved by a superabsorbent comprising fluorofamide. Further, a process for its production has been found. Yet further, products for absorbing water that comprise that superabsorbent have been found.

The superabsorbent of this invention generally comprises fluorofamide in an amount of at least 10 wt.-ppm, preferably at least 50 wt.-ppm, more preferably at least 100 wt.-ppm and generally at most 5 000 wt.-ppm, preferably at most 3 000 wt.-ppm and more preferably at most 1 000 wt.-ppm, in each case based on the total weight of material.

The amount of fluorofamide to be added is chosen to impart the desired odour-control effect. A superabsorbent that, on itself, shows less odour-formation (as non-limiting example an “acidic” superabsorbent having a comparatively lower degree of neutralisation (described below) than other superabsorbents) needs less fluorofamide. Higher amounts of fluorofamide than stated above may be necessary in some non-standard cases to impart even higher odour control effect, if so desired. The amounts above are sufficient to impart the desired odour-control effect in standard cases such as in personal hygiene products comprising superabsorbent that are used to absorb body fluids. It is obvious that odour control beyond the time at which personal hygiene products have to be changed for capacity reasons is typically not necessary, but of course it is possible to add higher amounts of fluorofamide. Lower amounts than those stated above may be sufficient in other non-standard cases where less odour-control effect is necessary.

A particular advantage of the superabsorbent of the present invention is that it is no biocide since fluorofamide is no biocide.

The superabsorbent in the present invention is a superabsorbent capable of absorbing and retaining amounts of water equivalent to many times its own weight under a certain pressure. Preferably, the superabsorbent is a crosslinked polymer based on partially neutralised acrylic acid and more preferably it is surface postcrosslinked. A “superabsorbent” can also be a mixture of chemically different individual superabsorbents in that it is not so much the chemical composition which matters as the superabsorbing properties.

Processes for producing superabsorbents, including surface-postcrosslinked superabsorbents, are known. Most synthetic superabsorbents on the market today are obtained by a process comprising polymerisation of a monomer solution comprising:

a) at least one ethylenically unsaturated monomer which bears acid groups and may be at least partly neutralised,

b) at least one crosslinker,

c) at least one initiator,

d) optionally one or more ethylenically unsaturated monomers copolymerisable with the monomers specified under a) and

e) optionally one or more water-soluble polymers.

The process typically further comprises drying, grinding, classifying and/or surface postcrosslinking the resulting polymer.

The monomers a) are preferably water-soluble, i.e. the solubility in water at 23° C. is typically at least 1 g/100 g of water, preferably at least 5 g/100 g of water, more preferably at least 25 g/100 g of water, most preferably at least 35 g/100 g of water.

Suitable monomers a) are, for example, ethylenically unsaturated carboxylic acids, such as acrylic acid, methacrylic acid and itaconic acid. Particularly preferred monomers are acrylic acid and methacrylic acid. Very particular preference is given to acrylic acid.

Further suitable monomers a) are, for example, ethylenically unsaturated sulfonic acids, such as styrenesulfonic acid and 2-acrylamido-2-methylpropanesulfonic acid (AMPS).

Impurities can have a considerable influence on the polymerisation. The raw materials used should therefore have a maximum purity. It is therefore often advantageous to specially purify the monomers a). Suitable purification processes are described, for example, in WO 2002/055469 A1, WO 2003/078378 A1 and WO 2004/035514 A1. A suitable monomer a) is, for example, acrylic acid purified according to WO 2004/035514 A1 comprising 99.8460% by weight of acrylic acid, 0.0950% by weight of acetic acid, 0.0332% by weight of water, 0.0203% by weight of propionic acid, 0.0001% by weight of furfurals, 0.0001% by weight of maleic anhydride, 0.0003% by weight of diacrylic acid and 0.0050% by weight of hydroquinone monomethyl ether.

The proportion of acrylic acid and/or salts thereof in the total amount of monomers a) is preferably at least 50 mol %, more preferably at least 90 mol %, most preferably at least 95 mol %.

The monomers a) typically comprise polymerisation inhibitors, preferably hydroquinone half ethers, as storage stabilisers.

The monomer solution comprises preferably up to 250 ppm by weight, preferably at most 130 ppm by weight, more preferably at most 70 ppm by weight, preferably at least 10 ppm by weight, more preferably at least 30 ppm by weight, especially around 50 ppm by weight, of hydroquinone half ether, based in each case on the unneutralised monomer a). For example, the monomer solution can be prepared by using an ethylenically unsaturated monomer bearing acid groups with an appropriate content of hydroquinone half ether.

Preferred hydroquinone half ethers are hydroquinone monomethyl ether (MEHQ) and/or alpha-tocopherol (vitamin E).

Suitable crosslinkers b) are compounds having at least two groups suitable for crosslinking. Such groups are, for example, ethylenically unsaturated groups which can be polymerised free-radically into the polymer chain, and functional groups which can form covalent bonds with the acid groups of the monomer a). In addition, polyvalent metal salts which can form coordinate bonds with at least two acid groups of the monomer a) are also suitable as crosslinkers b).

Crosslinkers b) are preferably compounds having at least two polymerisable groups which can be polymerised free-radically into the polymer network. Suitable crosslinkers b) are, for example, ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane, as described in EP 0 530 438 A1, di- and triacrylates, as described in EP 0 547 847 A1, EP 0 559 476 A1, EP 0 632 068 A1, WO 93/21237 A1, WO 2003/104299 A1, WO 2003/104300 A1, WO 2003/104301 A1 and DE 103 31 450 A1, mixed acrylates which, as well as acrylate groups, comprise further ethylenically unsaturated groups, as described in DE 103 31 456 A1 and DE 103 55 401 A1, or crosslinker mixtures, as described, for example, in DE 195 43 368 A1, DE 196 46 484 A1, WO 90/15830 A1 and WO 2002/032962 A2.

Preferred crosslinkers b) are pentaerythrityl triallyl ether, tetraalloxyethane, methylenebismethacrylamide, 15-tuply ethoxylated trimethylolpropane triacrylate, polyethylene glycol diacrylate, trimethylolpropane triacrylate and triallylamine.

Very particularly preferred crosslinkers b) are the polyethoxylated and/or propoxylated glycerols which have been esterified with acrylic acid or methacrylic acid to give di- or triacrylates, as described, for example, in WO 2003/104301 A1. Di- and/or triacrylates of 3- to 10-tuply ethoxylated glycerol are particularly advantageous. Very particular preference is given to di- or triacrylates of 1- to 5-tuply ethoxylated and/or propoxylated glycerol. Most preferred are the triacrylates of 3- to 5-tuply ethoxylated and/or propoxylated glycerol, especially the triacrylate of 3-tuply ethoxylated glycerol.

The amount of crosslinker b) is preferably from 0.05 to 1.5% by weight, more preferably from 0.1 to 1% by weight, most preferably from 0.3 to 0.6% by weight, based in each case on monomer a). With rising crosslinker content, the centrifuge retention capacity (CRC) falls and the absorption under a pressure of 21.0 g/cm² (AUL 0.3 psi) passes through a maximum.

The initiators c) may be all compounds which generate free radicals under the polymerisation conditions, for example thermal initiators, redox initiators, photoinitiators. Suitable redox initiators are sodium peroxodisulfate/ascorbic acid, hydrogen peroxide/ascorbic acid, sodium peroxodisulfate/sodium bisulfite and hydrogen peroxide/sodium bisulfite. Preference is given to using mixtures of thermal initiators and redox initiators, such as sodium peroxodisulfate/hydrogen peroxide/ascorbic acid. The reducing component used is, however, preferably a mixture of the sodium salt of 2-hydroxy-2-sulfinatoacetic acid, the disodium salt of 2-hydroxy-2-sulfonatoacetic acid and sodium bisulfite. Such mixtures are obtainable as Brüggolite® FF6 and Brüggolite® FF7 (Brüggemann Chemicals; Heilbronn; Germany).

Ethylenically unsaturated monomers d) copolymerisable with the ethylenically unsaturated monomers a) bearing acid groups are, for example, acrylamide, methacrylamide, hydroxyethyl acrylate, hydroxyethyl methacrylate, dimethylaminoethyl methacrylate, dimethylaminoethyl acrylate, dimethylaminopropyl acrylate, diethylaminopropyl acrylate, dimethylaminoethyl methacrylate, diethylaminoethyl methacrylate.

The water-soluble polymers e) used may be polyvinyl alcohol, polyvinylpyrrolidone, starch, starch derivatives, modified cellulose, such as methylcellulose or hydroxyethylcellulose, gelatin, polyglycols or polyacrylic acids, preferably starch, starch derivatives and modified cellulose.

Typically, an aqueous monomer solution is used. The water content of the monomer solution is preferably from 40 to 75% by weight, more preferably from 45 to 70% by weight, most preferably from 50 to 65% by weight. It is also possible to use monomer suspensions, i.e. monomer solutions with excess monomer a), for example sodium acrylate. With rising water content, the energy requirement in the subsequent drying rises, and, with falling water content, the heat of polymerisation can only be removed inadequately.

For optimal action, the preferred polymerisation inhibitors require dissolved oxygen. Before the polymerisation, the monomer solution can therefore be freed of dissolved oxygen, and the polymerisation inhibitor present in the monomer solution can be deactivated, by inertisation. A comfortable method for inertisation is passing a flow of an inert gas through the monomer solution, preferably nitrogen or carbon dioxide. The oxygen content of the monomer solution is preferably lowered before the polymerisation to less than 1 ppm by weight, more preferably to less than 0.5 ppm by weight, most preferably to less than 0.1 ppm by weight.

Suitable reactors are, for example, kneading reactors or belt reactors. In the kneader, the polymer gel formed in the polymerisation of an aqueous monomer solution or suspension is comminuted continuously by, for example, contrarotatory stirrer shafts, as described in WO 2001/038402 A1. Polymerisation on a belt is described, for example, in DE 38 25 366 A1 and U.S. Pat. No. 6,241,928. Polymerisation in a belt reactor forms a polymer gel, which has to be comminuted in a further process step, for example in an extruder or kneader.

However, it is also possible to generate droplets of an aqueous monomer solution and to polymerise the droplets obtained in a heated carrier gas stream. This allows the process steps of polymerisation and drying to be combined, as described in WO 2008/040715 A2 and WO 2008/052971 A1.

The acid groups of the resulting polymer gels have typically been partially neutralised. Neutralisation is preferably carried out at the monomer stage. This is typically done by mixing in the neutralising agent as an aqueous solution or preferably also as a solid. The degree of neutralisation is preferably from 25 to 95 mol %, more preferably from 30 to 80 mol %, most preferably from 40 to 75 mol %, for which the customary neutralising agents can be used, preferably alkali metal hydroxides, alkali metal oxides, alkali metal carbonates or alkali metal hydrogencarbonates and also mixtures thereof. Instead of alkali metal salts, it is also possible to use ammonium salts. Particularly preferred alkali metals are sodium and potassium, but very particular preference is given to sodium hydroxide, sodium carbonate or sodium hydrogencarbonate and also mixtures thereof.

However, it is also possible to carry out neutralisation after the polymerisation, at the stage of the polymer gel formed in the polymerisation. It is also possible to neutralise up to 40 mol %, preferably from 10 to 30 mol % and more preferably from 15 to 25 mol % of the acid groups before the polymerisation by adding a portion of the neutralising agent actually to the monomer solution and setting the desired final degree of neutralisation only after the polymerisation, at the polymer gel stage. When the polymer gel is neutralised at least partly after the polymerisation, the polymer gel is preferably comminuted mechanically, for example by means of an extruder, in which case the neutralising agent can be sprayed, sprinkled or poured on and then carefully mixed in. To this end, the gel mass obtained can be repeatedly extruded for homogenisation.

The polymer hydrogel is then typically dried with a belt dryer until the residual moisture content is preferably from 0.5 to 15% by weight, more preferably from 1 to 10% by weight, most preferably from 2 to 8% by weight. In the case of too high a residual moisture content, the dried polymer gel has too low a glass transition temperature Tg and can be processed further only with difficulty. In the case of too low a residual moisture content, the dried polymer gel is too brittle and, in the subsequent comminution steps, undesirably large amounts of polymer particles with an excessively low particle size are obtained (fines). The solids content of the gel before the drying is preferably from 25 to 90% by weight, more preferably from 35 to 70% by weight, most preferably from 40 to 60% by weight. Optionally, it is, however, also possible to use a fluidised bed dryer or a paddle dryer for the drying operation.

The dried hydrogel (which is no longer a gel (even though often still called that) but a dry polymer having superabsorbing properties, which comes within the term “super-absorbent”) is typically ground and sieved to produce a particulate superabsorbent “base polymer” of the desired particle size distribution. Useful grinding apparatus typically including single or multistage roll mills, pin mills, hammer mills, cutting mills or swing mills.

The mean particle size of the polymer particles collected as the product fraction is preferably at least 200 μm, more preferably from 250 to 600 μm, very particularly from 300 to 500 μm.

For economic reasons, it is desirable to select conditions of grinding and other steps of mechanical treatment to achieve a proportion of particles with a particle size of at least 150 μm of preferably at least 90% by weight, more preferably at least 95% by weight, most preferably at least 98% by weight.

Polymer particles with too small a particle size lower the permeability (SFC). The proportion of excessively small polymer particles (fines) should therefore be small.

Excessively small polymer particles are therefore typically removed and recycled into the process. This is preferably done before, during or immediately after the polymerisation, i.e. before the drying of the polymer gel. The excessively small polymer particles can be moistened with water and/or aqueous surfactant before or during the recycling.

It is also possible to remove excessively small polymer particles in later process steps, for example after the surface postcrosslinking or another coating step. In this case, the excessively small polymer particles recycled are surface postcrosslinked or coated in another way, for example with fumed silica.

When a kneading reactor is used for the polymerisation, the excessively small polymer particles are preferably added during the last third of the polymerisation.

When the excessively small polymer particles are added at a very early stage, for example actually to the monomer solution, this lowers the centrifuge retention capacity (CRC) of the resulting water-absorbing polymer particles. However, this can be compensated, for example, by adjusting the amount of crosslinker b) used.

When the excessively small polymer particles are added at a very late stage, for example not until within an apparatus connected downstream of the polymerisation reactor, for example an extruder, the excessively small polymer particles can be incorporated into the resulting polymer gel only with difficulty. Excessively small polymer particles which have been insufficiently incorporated, however, become detached again from the dried polymer gel during the grinding, and are therefore removed again in the classification and increase the amount of excessively small polymer particles to be recycled.

For economic reasons, it is desirable to select conditions of grinding and other steps of mechanical treatment to achieve a proportion of particles with a particle size of at most 850 μm of preferably at least 90% by weight, more preferably at least 95% by weight, most preferably at least 98% by weight.

Advantageously, the proportion of polymer particles with a particle size of at most 600 μm is preferably at least 90% by weight, more preferably at least 95% by weight, most preferably at least 98% by weight.

Polymer particles with too great a particle size lower the swell rate. The proportion of excessively large polymer particles should therefore likewise be small.

Excessively large polymer particles are therefore typically removed and recycled into the grinding of the dried polymer gel.

To further improve the properties, the base polymer particles are optionally surface postcrosslinked. Suitable surface postcrosslinkers are compounds which comprise groups which can form covalent bonds with at least two carboxylate groups of the polymer particles. Suitable compounds are, for example, polyfunctional amines, polyfunctional amido amines, polyfunctional epoxides, as described in EP 0 083 022 A2, EP 0 543 303 A1 and EP 0 937 736 A2, di- or polyfunctional alcohols, as described in DE 33 14 019 A1, DE 35 23 617 A1 and EP 0 450 922 A2, or β-hydroxyalkylamides, as described in DE 102 04 938 A1 and U.S. Pat. No. 6,239,230.

Additionally described as suitable surface postcrosslinkers are cyclic carbonates in DE 40 20 780 C1, 2-oxazolidone and its derivatives, such as 2-hydroxyethyl-2-oxazolidone in DE 198 07 502 A1, bis- and poly-2-oxazolidinones in DE 198 07 992 C1, 2-oxotetrahydro-1,3-oxazine and its derivatives in DE 198 54 573 A1, N-acyl-2-oxazolidones in DE 198 54 574 A1, cyclic ureas in DE 102 04 937 A1, bicyclic amide acetals in DE 103 34 584 A1, oxetanes and cyclic ureas in EP 1 199 327 A2 and morpholine-2,3-dione and its derivatives in WO 2003/031482 A1.

Preferred surface postcrosslinkers are glycerol, ethylene carbonate, ethylene glycol diglycidyl ether, reaction products of polyamides with epichlorohydrin, and mixtures of propylene glycol and 1,4-butanediol.

Very particularly preferred surface postcrosslinkers are 2-hydroxyethyloxazolidin-2-one, oxazolidin-2-one and 1,3-propanediol.

In addition, it is also possible to use surface postcrosslinkers which comprise additional polymerisable ethylenically unsaturated groups, as described in DE 37 13 601 A1.

The amount of surface postcrosslinker is preferably from 0.001 to 2% by weight, more preferably from 0.02 to 1% by weight, most preferably from 0.05 to 0.2% by weight, based in each case on the polymer particles.

The surface postcrosslinking is typically performed in such a way that a solution of the surface postcrosslinker is sprayed onto the dried polymer particles. After the spraying, the polymer particles coated with surface postcrosslinker are dried thermally, and the surface postcrosslinking reaction can take place either before or during the drying.

The spraying of a solution of the surface postcrosslinker is preferably performed in mixers with moving mixing tools, such as screw mixers, disk mixers and paddle mixers. Particular preference is given to horizontal mixers such as paddle mixers, very particular preference to vertical mixers. The distinction between horizontal mixers and vertical mixers is made by the position of the mixing shaft, i.e. horizontal mixers have a horizontally mounted mixing shaft and vertical mixers a vertically mounted mixing shaft. Suitable mixers are, for example, horizontal Pflugschar® mixers (Gebr. Lödige Maschinenbau GmbH; Paderborn; Germany), Vrieco-Nauta Continuous Mixers (Hosokawa Micron BV; Doetinchem; the Netherlands), Processall Mixmill Mixers (Processall Incorporated; Cincinnati; US) and Schugi Flexomix® (Hosokawa Micron BV; Doetinchem; the Netherlands). However, it is also possible to spray the surface postcrosslinker solution into a fluidised bed of base polymer.

The surface postcrosslinkers are typically used in the form of an aqueous solution. The penetration depth of the surface postcrosslinker into the polymer particles can be adjusted via the content of nonaqueous solvent and total amount of solvent.

When exclusively water is used as the solvent, a surfactant is advantageously added. This improves the wetting behavior and reduces the tendency to form lumps. However, preference is given to using solvent mixtures, for example isopropanol/water, 1,3 propanediol/water and propylene glycol/water, where the mixing ratio in terms of mass is preferably from 20:80 to 40:60.

The thermal drying is preferably carried out in contact dryers, more preferably paddle dryers, most preferably disk dryers. Suitable dryers are, for example, Hosokawa Bepex® Horizontal Paddle Dryers (Hosokawa Micron GmbH; Leingarten; Germany), Hosokawa Bepex® Disc Dryers (Hosokawa Micron GmbH; Leingarten; Germany) and Nara Paddle Dryers (NARA Machinery Europe; Frechen; Germany). Moreover, it is also possible to use fluidised bed dryers.

The drying can be effected in the mixer itself, by heating the jacket or blowing in warm air. Equally suitable is a downstream dryer, for example a shelf dryer, a rotary tube oven or a heatable screw. It is particularly advantageous to mix and dry in a fluidised bed dryer.

Preferred drying temperatures are in the range from 100 to 250° C., preferably from 120 to 220° C., more preferably from 130 to 210° C., most preferably from 150 to 200° C. The preferred residence time at this temperature in the reaction mixer or dryer is preferably at least 10 minutes, more preferably at least 20 minutes, most preferably at least 30 minutes, and typically at most 60 minutes.

Quite usually, but not necessarily, water-soluble polyvalent metal salts comprise bi- or more highly valent (“polyvalent”) metal cations capable of reacting with the acid groups of the polymer to form complexes are added. Examples of polyvalent cations are or metal cations such as Mg²⁺, Ca²⁺, Al³⁺, Sc³⁺, Ti⁴⁺, Mn²⁺, Fe^{2+/3+}, Co²⁺, Ni²⁺, Cu²⁺, Zn²⁺, Y³⁺, Zr⁴⁺, La³⁺, Ce⁴⁺, Hf⁴⁺, and Au³⁺. Preferred metal cations are Mg²⁺, Ca²⁺, Al³⁺, Ti⁴⁺, Zr⁴⁺ and La³⁺, and particularly preferred metal cations are Al³⁺, Ti⁴⁺ and Zr⁴⁺. The metal cations can be used not only alone but also in admixture with each other. Of the metal cations mentioned, any metal salt can be used that has sufficient solubility in the solvent to be used. Metal salts with weakly complexing anions such as for example chloride, nitrate and sulphate, hydrogen sulphate, carbonate, hydrogen carbonate, nitrate, phosphate, hydrogen phosphate, dihydrogen phosphate and carboxylate, such as acetate and lactate, are particularly suitable. It is particularly preferred to use aluminum sulfate.

The treatment of the superabsorbent polymer with solution of a polyvalent cation is car-ried out in the same way as that with surface postcrosslinker, including the selective drying step. Useful solvents for the metal salts include water, alcohols, DMF, DMSO and also mixtures thereof. Particular preference is given to water and water-alcohol mixtures such as for example water-methanol, water-1,2-propanediol, water-2-propanol and water-1,3-propanediol.

In a preferred embodiment of the present invention, the complexing agent is applied to a superabsorbent that is surface crosslinked, or concurrently with surface crosslinking, or partly simultaneously and partly after surface crosslinking. For example, a suitable method of applying a complexing agent is applying a polyvalent metal cation such as Al³⁺ concurrently with a surface crosslinker.

The surface-crosslinked superabsorbent produced using the process of the instant inventions is optionally ground and/or sieved in a conventional manner. Grinding is typically not necessary, but the sieving out of agglomerates which are formed or undersize is usually advisable to set the desired particle size distribution for the product. Agglomerates and undersize are either discarded or preferably returned into the process in a conventional manner and at a suitable point; agglomerates after comminution.

To improve some properties such as brittleness, the surface postcrosslinked polymer particles may be moistened. Moistening is carried out preferably at from 30 to 80° C., more preferably at from 35 to 70° C. and most preferably at from 40 to 60° C. At excessively low temperatures, the water-absorbing polymer particles tend to form lumps, and, at higher temperatures, water already evaporates noticeably. The amount of water used for subsequent moistening is preferably from 1 to 10% by weight, more preferably from 2 to 8% by weight and most preferably from 3 to 5% by weight. The subsequent moistening increases the mechanical stability of the polymer particles and reduces their tendency to static charging.

If the superabsorbent is produced by another method, some of the process steps described above may be unnecessary. For example, emulsion or droplet polymerisation produces particulate superabsorbents that may not need grinding or classification or surface crosslinking. Further, droplet polymerisation is typically performed in reactors that also dry the product due to the gas streams typically necessary to conduct droplet polymerisation, so a separate drying step may not be necessary. The method of producing the superabsorbent plays no role in this invention and any particulate superabsorbent produced in any manner may be treated according to this invention.

After any drying, curing or other heating step, it may be advantageous but not absolutely necessary to cool the product. Cooling can be carried out continuously or discontinuously, conveniently by conveying the product continuously into a cooler downstream of the dryer. Any apparatus known for removing heat from pulverulent solids can be used, in particular any apparatus mentioned above as a drying apparatus, provided it is supplied not with a heating medium but with a cooling medium such as for example with cooling water, so that heat is not introduced into the superabsorbent via the walls and, depending on the design, also via the stirrer elements or other heat-exchanging surfaces, but removed from the superabsorbent. Preference is given to the use of coolers in which the product is agitated, i.e., cooled mixers, for example shovel coolers, disk coolers or paddle coolers, for example Nara® or Bepex® coolers. The superabsorbent can also be cooled in a fluidised bed by blowing a cooled gas such as cold air into it. The cooling conditions are set such that a superabsorbent having the temperature desired for further processing is obtained. Typically, the average residence time in the cooler will be in general at least 1 minute, preferably at least 3 minutes and more preferably at least 5 minutes and also in general not more than 6 hours, preferably 2 hours and more preferably not more than 1 hour, and cooling performance will be determined such that the product obtained has a temperature of generally at least 0° C., preferably at least 10° C. and more preferably at least 20° C. and also generally not more than 100° C., preferably not more than 80° C. and more preferably not more than 60° C.

According to this invention, fluorofamide is added to the superabsorbent. In the simplest and most preferred form, fluorofamide is added as a substance to the superabsorbent at any stage of the process for its production. One embodiment of the invention is adding fluorofamide to the monomer solution. Another embodiment is adding fluorofamide after polymerisation, for example prior to, during or after a neutralisation step following polymerisation. It is generally preferred, however, to add fluorofamide after drying the polymerised hydrogel. In one embodiment, the superabsorbent is contacted with a solution or slurry of fluorofamide in a solvent or dispersion medium. The solution or slurry may, however, comprise other desired components such as for example, but not limited to other additive known for superabsorbents.

The solution or slurry can be added in any known way. Spraying the solution or slurry onto the superabsorbent is preferred. The solution or slurry may be added in the same type of apparatus that is used for contacting the superabsorbent with the surface crosslinking solution. In a convenient way, the solution or slurry is added by spraying on the superabsorbent in a final cooler.

Preferably, the solution or slurry is added concurrently with any other chosen additive following any surface crosslinking or complexing step. In this context, “concurrently” means “in the very same piece of equipment”, but does not necessarily mean “through the very same nozzle or set of nozzles”.

It is preferred to add the fluorofamide without any subsequent heating. In other words, it is preferred to add the fluorofamide after any drying, curing or other heating step in which the temperature of the superabsorbent is purposefully increased.

Useful solvents or dispersants for fluorofamide include those that solve or disperse enough fluorofamide to add to the superabsorbent at total solvent or dispersant amounts that do not negatively influence the process. Examples of useful solvents are polar solvents. Water or dimethyl sulfoxide (“DMSO”), alcohols, DMF, or mixtures thereof, including, but not limited to water-alcohol mixtures such as water-methanol, water-1,2-propanediol, water-2-propanol and water-1,3-propanediol and propylene glycol/water, where the mixing ratio in terms of mass is preferably from 20:80 to 40:60 may be used.

The concentration of the solution or slurry is not particularly critical. The main criterion for the concentration is to have a solution or slurry that can be processed in the chosen equipment. When spraying the solution or slurry, the solution or slurry needs to be pumpable to and sprayable from the chosen nozzle. It is preferred that the solution or slurry essentially consists of fluorofamide and solvent or dispersant and it is more preferred that it consists of fluorofamide and solvent or dispersant.

Optionally, the superabsorbent is provided with further customary additives and auxiliary materials to influence storage or handling properties. Examples thereof are colorations, opaque additions to improve the visibility of swollen gel, which is desirable in some applications, surfactants, de-dusting agents, colour stabilisers, flowability aids, anticaking additives or the like. These additives and auxiliary materials can each be added in separate processing steps, but one convenient method may be to add them to the superabsorbent in the cooler, for example by spraying the superabsorbent with a solution or adding them in finely divided solid or in liquid form, if this cooler provides sufficient mixing quality.

The inventive water-absorbing polymer particles have a moisture content of typically 0 to 15% by weight, preferably 0.2 to 10% by weight, more preferably 0.5 to 8% by weight, most preferably 1 to 5% by weight, and/or a centrifuge retention capacity (CRC) of typically at least 20 g/g, preferably at least 26 g/g, more preferably at least 28 g/g, most preferably at least 30 g/g, and/or an absorption under a pressure of 49.2 g/cm² (AUL 0.7 psi) of typically at least 12 g/g, preferably at least 16 g/g, more preferably at least 18 g/g, most preferably at least 20 g/g, and/or a saline flow conductivity (SFC) of typically at least 20·10⁻⁷ cm³ s/g, preferably at least 40·10⁻⁷ cm³ s/g, more preferably at least 50·10⁻⁷ cm³ s/g, most preferably at least 60·10⁻⁷ cm³ s/g.

The centrifuge retention capacity (CRC) of the water-absorbing polymer particles is typically less than 60 g/g. The absorption under a pressure of 49.2 g/cm² (AUL 0.7 psi) of the water-absorbing polymer particles is typically less than 35 g/g. The saline flow conductivity (SFC) of the water-absorbing polymer particles is typically less than 200·10⁻⁷ cm³ s/g.

We have further found water-absorbing products, in particular hygiene articles comprising the superabsorbent of the present invention. Hygiene articles in accordance with the present invention are for example those intended for use in mild or severe incontinence, such as for example inserts for severe or mild incontinence, incontinence briefs, also diapers, training pants for babies and infants or else feminine hygiene articles such as liners, sanitary napkins or tampons. Hygiene articles of this kind are known. The hygiene articles of the present invention differ from known hygiene articles in that they comprise the superabsorbent of the present invention. We have also found a process for producing water-absorbing products, in particular hygiene articles, this process comprising adding at least one superabsorbent of the present invention to the in the manufacture of the water-absorbing-product in particular hygiene article in question during its manufacture. Processes for producing water-absorbing products, in particular hygiene articles comprising superabsorbent are otherwise known.

The present invention further provides for the use of the composition of the present invention in training pants for children, shoe inserts and other hygiene articles to absorb bodily fluids. The composition of the present invention can also be used in other technical and industrial fields where liquids, in particular water or aqueous solutions, are absorbed. These fields are for example storage, packaging, transportation (as constituents of packaging material for water- or moisture-sensitive articles, for example for flower transportation, also as protection against mechanical impacts); animal hygiene (in cat litter); food packaging (transportation of fish, fresh meat; absorption of water, blood in fresh fish or meat packs); medicine (wound plasters, water-absorbing material for burn dressings or for other weeping wounds), cosmetics (carrier material for pharmachemicals and medicaments, rheumatic plasters, ultrasonic gel, cooling gel, cosmetic thickeners, sun protection); thickeners for oil-in-water and water-in-oil emulsions; textiles (moisture regulation in textiles, shoe inserts, for evaporative cooling, for example in protective clothing, gloves, headbands); chemical engineering applications (as a catalyst for organic reactions, to immobilise large functional molecules such as enzymes, as adhesion agent in relation to agglomerations, heat storage media, filter aids, hydrophilic component in polymeric laminates, dispersants, superplasticisers); as auxiliaries in powder injection moulding, in building construction and engineering (installation, in loam-based renders, as a vibration-inhibiting medium, auxiliaries in tunnel excavations in water-rich ground, cable sheathing); water treatment, waste treatment, water removal (deicing agents, reusable sandbags); cleaning; agritech (irrigation, retention of melt water and dew deposits, composting additive, protection of forests against fungal/insect infestation, delayed release of active components to plants); for firefighting or for fire protection; coextrusion agents in thermoplastic polymers (for example to hydrophilise multilayered films); production of films and thermoplastic mouldings able to absorb water (for example rain and dew water storage films for agriculture; superabsorbent-containing films for keeping fruit and vegetables fresh which are packed in moist films; superabsorbent-polystyrene coextrudates, for example for food packaging such as meat, fish, poultry, fruit and vegetables); or as carrier substance in formulations of active components (pharma, crop protection).

### Test Methods

The standard test methods referred to as “WSP” described below are described in: “Standard Test Methods for the Nonwovens Industry”, 2005 edition, published jointly by the Worldwide Strategic Partners EDANA (European Disposables and Nonwovens Association, Avenue Eugéne Plasky, 157, 1030 Brussels, Belgium, www.edana.org) and INDA (Association of the Nonwoven Fabrics Industry, 1100 Crescent Green, Suite 115, Cary, N.C. 27518, U.S.A., www.inda.org). This publication is obtainable both from EDANA and from INDA.

The measurements should, unless stated otherwise, be carried out at an ambient temperature of 23±2° C. and a relative air humidity of 50±10%. The water-absorbing polymer particles are mixed thoroughly before the measurement.

### Saline Flow Conductivity (“SFC”)

The saline flow conductivity (SFC) of a swollen gel layer under a pressure of 0.3 psi (2070 Pa) is, as described in EP 0 640 330 A1 (page 19, line 13 to page 21, line 35), determined as the gel layer permeability of a swollen gel layer of water-absorbing polymer particles, with modification of the apparatus described in FIG. 8 in that the glass frit (40) is not used, the plunger (39) consists of the same plastic material as the cylinder (37), and now has 21 bores of equal size distributed homogeneously over the entire contact area. The procedure and evaluation of the measurement remain unchanged from EP 0 640 330 A1. The flow is detected automatically.

The saline flow conductivity (SFC) is calculated as follows:

SFC [cm³ s/g]=(Fg(t=0)×L0)/(d×A×WP)

where Fg(t=0) is the flow of NaCl solution in g/s, which is obtained using a linear regression analysis of the Fg(t) data of the flow determinations by extrapolation to t=0, L0 is the thickness of the gel layer in cm, d is the density of the NaCl solution in g/cm³, A is the area of the gel layer in cm², and WP is the hydrostatic pressure over the gel layer in dyn/cm².

### Centrifuge Retention Capacity (“CRC”)

The centrifuge retention capacity (CRC) is determined by test method No. WSP 214.2-05 “Centrifuge Retention Capacity”.

Absorption Under a Pressure of 21.0 g/cm² (“AUL 0.3 psi”)

The absorption under a pressure of 49.2 g/cm² (commonly referred to as “AUL 0.3 psi”) is determined by test method No. WSP 242.2-05 “Absorption under Pressure”

Absorption Under a Pressure of 49.2 g/cm² (“AUL 0.7 psi”)

The absorption under a pressure of 49.2 g/cm² (commonly referred to as “AUL0.7 psi”) is determined by test method No. WSP 242.2-05 “Absorption under Pressure”, however, with a pressure setting of 49.2 g/cm² (AUL0.7 psi) instead of 21.0 g/cm² (that corresponds to the AUL0.3 psi).

### Water or Moisture Content

Water or moisture content is determined by test method No. WSP 230.2-05 “Moisture Content”.

### Odour Inhibition

DSM1 medium (Deutsche Sammlung von Mikroorganismen and Zellkulturen GmbH, Inhoffenstraβe 7 B, 38124 Braunschweig, Germany) was prepared from 5.0 g/l peptone from meat (Merck KGaA, Frankfurter Strasse 250, 64293 Darmstadt, Germany; Art. Nr. 1.07214) and 3.0 g/l meat extract (Merck; Art. Nr. 1103979) and set to pH=7.0. 50 ml DSM1 medium was inoculated with proteus mirabilis ATCC 14153 to OD=0.1 and incubated in a stirred 250 ml Erlenmeyer flask with baffle for 15 hours at 37° C. and 220 rpm. The obtained stock solution had a cell density of about 10⁹ cfu/ml (OD=2.0-2.5).

Synthetic urine was prepared from 25 g/l urea (sterile filtrated), 9.0 g/l sodium chloride, 1 g/l peptone from meat and 1 g/l meat extract. The synthetic urine was steam autoclaved prior to adding the concentrated sterile filtrated urea solution.

The amount of superabsorbent necessary to absorb 50 ml of synthetic urine (calculated from the centrifuge retention capacity) was placed in a 125 ml steam autoclaved polypropylen histology beaker. 50 ml synthetic urine were inoculated with 50 μl of the stock solution, corresponding to a total concentration of about 10⁶ cfu/ml, mixed with the superabsorbent, and the lid, equipped with a diffusion tube (“Dräger-Röhrchen® Ammoniak 20/a-D”, available from Dragerwerk AG & Co. KGaA; Lübeck; Germany; item no. 8101301), was screwed on. Ammonia generation was monitored for 72 hours at 37° C. The time needed to reach the detection limit for ammonia (which is 1 500 ppm h) was recorded.

### EXAMPLES

### Example 1

### Preparation of Base Polymer

A double-wall 10 l glass reactor with mechanical stirring was initially charged with 4691 g of a 37.3% by weight sodium acrylate solution which had been filtered through activated carbon beforehand, and 526 g of water. With stirring and simultaneous cooling, 560 g of acrylic acid were metered in gradually. After bubbling nitrogen through for 30 minutes, 6.59 g of triply ethoxylated glyceryl triacrylate and 9.2 g of a 30% by weight solution of sodium persulfate in water were added, and the mixture was stirred for a further minute. In the course of this, the reaction mixture was cooled such that the temperature at no time exceeded 35° C. and was approx. 20° C. toward the end. The reaction mixture was subsequently transferred by means of a pump into an IKA® HKS horizontal kneader (capacity 10 l, available from IKA®-Werke GmbH & Co. KG, 79219 Staufen, Germany) which had been preheated to 60° C. and was purged with nitrogen gas. Finally, in the horizontal kneader, 4.7 g of a 1% by weight solution of ascorbic acid in water and 0.55 g of 3% by weight hydrogen peroxide were added with stirring in the horizontal kneader. The reactor jacket temperature was raised to 95° C. and, after 15 minutes of reaction time, the polymer gel formed was removed from the horizontal kneader. The polymer gel thus obtained was distributed on metal sheets with wire bases and dried in a forced air drying cabinet at 165° C. for 90 minutes. This was followed by comminution with an ultracentrifugal mill, and the product was screened to obtain the fraction from 150 to 850 μm. The base polymer thus prepared had a centrifuge retention capacity (CRC) of 38 g/g.

### Example 2

### Surface Crosslinking

1 kg of the water-absorbent polymer particles from in example 1 was pre-heated in a laboratory oven to 50° C. Upon the water-absorbent polymer particles reached the oven temperature the water-absorbent polymer particles were put into a laboratory Ploughshare® mixer with a heated jacket (model M 5; manufactured by Gebrüder Lödige Maschinenbau GmbH; Paderborn; Germany). The temperature was kept at 190° C. during the surface crosslinking. A surface cross linker solution was prepared by mixing 0.7 g 2-hydroxyethyl-2-oxazolidon, 0.7 g 1,3-propanediole, 11.5 g of 2-propanol, 0.2 g of sorbitan monolaurate and 13.85 g of deionised water. At a mixer speed of 450 rpm, the surface cross linker solution was added to the polymer powder over a two minute time period using a disposable syringe. The product was kept at 190° C. for 60 minutes at a mixer speed of 210 rpm. After cooling down of the mixer, the product was discharged.

### Example 3

### No Fluorofamide (Comparative)

20 g of the polymer obtained in example 2 were placed in a blender (Blender 8012 model 34BL99; Waring Laboratory; US), with a top piece made from stainless steel (inner diameter 8 cm, inner height 4 cm, tool diameter 7 cm, an opening to add substances in the lid at 1.3 cm distance from rim, baffle mounted on lid). The blender was operated at level 3 setting. 1.84 g dimethyl sulfoxide (“DMSO”) (Sigma-Aldrich, order No 4141857) were added slowly with a syringe equipped with a needle. The polymer obtained was designated S0.

### Example 4

### Fluorofamide Addition

Example 3 was repeated, however, 1.84 g of a solution of 102 mg Fluorofamide (Tocris Bioscience, 5B TO Centre Carbot Park, Bristol, BS 11 0QL; UK) in 10 ml DMSO (Sigma-Aldrich, order No 4141857) were used instead of DMSO. The polymer obtained was designated S1.

### Example 5

### Fluorofamide Addition

Example 4 was repeated, however, only 0.92 g of the fluorofamide solution were added. The polymer obtained was designated S2.

### Example 6

### Fluorofamide Coating

Example 4 was repeated, however, only 0.18 g of the fluorofamide solution were added. The polymer obtained was designated S3.

Polymers S0-S3 were subjected to the Odour Control Tests. The following table is a summary of the results:

| | Flurofamide content | Time to detection limit |
|---|---|---|
| Polymer | [wt.-ppm] | [hours] |
| S0 | 0 | 9.2 |
| S1 | 1000 | 35.8 |
| S2 | 500 | 27.0 |
| S3 | 100 | 15.3 |

The data show that even low amounts of fluorofamide are able to control odours on a timescale typical for use of one piece of personal hygiene product.

1. A superabsorbent comprising fluorofamide.

2. The superabsorbent of claim 1, comprising fluorofamide in an amount of 10 wt.-ppm to 5 000 wt.-ppm, based on the total weight of superabsorbent.

3. The superabsorbent of claim 2, comprising fluorofamide in an amount of 100 wt.-ppm to 1 000 wt.-ppm, based on the total weight of superabsorbent.

4. The superabsorbent of claim 1, based on a crosslinked, partly neutralized polyacrylic acid.

5. The superabsorbent of claim 1 that is surface crosslinked.

6. A process for producing the superabsorbent defined in claim 1, comprising polymerisation of a monomer solution comprising:
a) at least one ethylenically unsaturated monomer which bears an acid group and may be at least partly neutralised,
b) at least one crosslinker,
c) at least one initiator,
d) optionally one or more ethylenically unsaturated monomer copolymerisable with the monomer specified under a) and
e) optionally one or more water-soluble polymer, the process further optionally comprising drying, grinding, classifying, and/or surface postcrosslinking the resulting polymer, and adding fluorofamide prior to, during, or after polymerization, drying, and/or surface crosslinking.

7. The process of claim 6, wherein the fluorofamide is added after drying or, if the superabsorbent is surface crosslinked, after surface crosslinking.

8. A water-absorbing article comprising the superabsorbent of claim 1.

9. The water-absorbing article of claim 8 that is a hygiene article.

10. A process for producing a water-absorbing article of claim 8 comprising adding the superabsorbent to the water-absorbing article during its production.
